(19) United States
(12) Patent Application Publication
(10) Pub. No.: US 2026/0114844 A1
(43) Pub. Date: Apr. 30, 2026

(54) TREATMENT SUPPORT APPARATUS AND TREATMENT SUPPORT PROGRAM

(71) Applicant: FUJIFILM Corporation, Tokyo (JP)

(72) Inventors: Shuichi DOKKO, Tokyo (JP); Koichi ISHIDA, Tokyo (JP)

(73) Assignee: FUJIFILM Corporation, Tokyo (JP)

(21) Appl. No.: 19/366,523

(22) Filed: Oct. 23, 2025

(30) Foreign Application Priority Data
Oct. 28, 2024 (JP) 2024-189253

(51) Int. Cl. A61B 8/00; A61B 1/00; A61B 34/00; A61B 34/20; A61B 34/30; A61B 34/37; A61B 90/00

(52) U.S. Cl. CPC A61B 8/4245; A61B 1/00149; A61B 8/466; A61B 8/483; A61B 34/20; A61B 34/25; A61B 34/37; A61B 90/39; A61B 2034/2055; A61B 2034/301; A61B 2090/3983

(57) ABSTRACT

Volume data is formed by an ultrasound probe transmitting and receiving ultrasound to and from a treatment target. The volume data has coordinate information indicating each position of the volume data based on a position and an orientation of the ultrasound probe during ultrasound transmission and reception. A treatment tool position/orientation detection unit is configured to specify a position and an orientation of a treatment tool in a data space of the volume data in a case in which the treatment on the treatment target is performed. A reconstruction processing unit is configured to form a reconstructed ultrasound image from the volume data with reference to the position and the orientation of the treatment tool in the data space of the volume data. A display controller is configured to display the reconstructed ultrasound image on a display.

### CROSS-REFERENCE TO RELATED APPLICATION

This application claims the priority benefit of Japan application serial no. 2024-189253, filed on Oct. 28, 2024. The entirety of the above-mentioned patent application is hereby incorporated by reference herein and made a part of this specification.

### BACKGROUND OF THE INVENTION

### 1. Field of the Invention

The present specification discloses improvements in a treatment support apparatus and a treatment support program.

### 2. Description of the Related Art

In the related art, in a case in which a treatment target of a subject is treated using a treatment tool (for example, scissors or forceps), an ultrasound diagnostic apparatus may be used for the purpose of checking the treatment target during the treatment. An ultrasound diagnostic apparatus is an apparatus that transmits ultrasound from an ultrasound probe toward the subject, receives a reflected wave from the subject in the ultrasound probe, and performs various processes such as the formation of an ultrasound tomographic image representing a cross section in the subject, the formation of a Doppler image representing a flow velocity of a tissue component (blood or the like) in the subject, and various kinds of measurement based on a reception signal formed from the reflected wave.

In the related art, various techniques related to an ultrasound diagnostic apparatus used in treatment on a treatment target have been proposed.

For example, JP2013-255658A discloses an ultrasound diagnostic apparatus comprising: a region-of-interest (ROI) mark setting unit that sets a region of interest designated by an examiner on an ultrasound image; a camera that acquires a camera image by imaging a subject to which a subject AR mark is attached and an ultrasound probe to which an ultrasound probe AR mark is attached; a non-subject coordinate conversion unit that calculates a body surface curve approximating a body surface of the subject based on the subject AR mark in the camera image; an ultrasound probe coordinate conversion unit that calculates 3D coordinate information of the ultrasound probe based on the ultrasound probe AR mark in the camera image; a 3D processor that calculates a position of the region of interest on the body surface of the subject based on the region of interest, the body surface curve of the subject, and the 3D coordinate information of the ultrasound probe; and an ROI/camera image combining unit that displays a ROI projection mark indicating the position of the region of interest in a superimposed manner on the camera image.

In addition, JP7284868B discloses a surgical system comprising an ultrasound probe that is inserted into a body cavity of a subject from a trocar and that transmits and receives ultrasound to and from a target tissue, an endoscope that images the ultrasound probe or a knife as a surgical treatment tool and a surface of a target tissue to obtain an endoscopic image, a position specifying unit that specifies positions of the ultrasound probe and the knife in the endoscopic image, and a controller. In the surgical system, first, the position of the ultrasound probe in the endoscopic image is specified, and then a plurality of combinations, each including the position of the ultrasound probe and an ultrasound tomographic image at the position that is formed by transmitting and receiving the ultrasound with the ultrasound probe to and from the target tissue, are stored in a storage unit. In addition, in a case in which the knife is inserted into the body cavity instead of the ultrasound probe, the position of the knife in the endoscopic image is specified, and the ultrasound tomographic image corresponding to the specified position of the knife is displayed on a display unit.

Further, JP4999012B discloses a robot surgical system comprising a first robot arm that holds an ultrasound probe, a second robot arm that holds a surgical instrument, and a master input device for remotely operating a plurality of robot arms, in which the robot surgical system is operable in a first mode in which the second robot arm is fixed at a predetermined position and the first robot arm is moved in accordance with the operation of the master input device, or a second mode in which the second robot arm is moved in accordance with the operation of the master input device and the first robot arm is moved in accordance with a user command.

### SUMMARY OF THE INVENTION

The operator cannot see the internal structure of the treatment target unless the treatment target is excised. Here, it is desirable that, prior to a treatment such as excision, the operator can view the internal structure of the treatment target near the treatment tool. As a result, for example, in a case of excising the treatment target, the operator can determine, before the treatment, an excision direction (excision line) to be followed by the scissors.

An object of the treatment support apparatus disclosed in the present specification is to allow an operator to check an internal structure of a treatment target near a location in which the operator intends to perform treatment with a treatment tool.

A treatment support apparatus disclosed in the present specification comprises: a probe position/orientation detection unit configured to detect a position and an orientation of an ultrasound probe; a volume data acquisition unit configured to acquire volume data that is formed based on a reception signal obtained by transmitting and receiving ultrasound with the ultrasound probe to and from a treatment target prior to treatment on the treatment target, the volume data having coordinate information indicating each position of the volume data in a data space of the volume data based on the position and the orientation of the ultrasound probe; a treatment tool position/orientation detection unit configured to specify a position and an orientation of a treatment tool for performing the treatment on the treatment target in the data space of the volume data in a case in which the treatment on the treatment target is performed; a reconstruction processing unit configured to form a reconstructed ultrasound image from the volume data with reference to the position and the orientation of the treatment tool in the data space of the volume data; and a display controller configured to display the reconstructed ultrasound image on a display unit.

The probe position/orientation detection unit may be configured to, based on a camera image during transmission and reception obtained by imaging a probe detection mark attached to the ultrasound probe and a target detection mark attached to the treatment target with a camera during ultrasound transmission and reception in which the ultrasound is transmitted to and received from the treatment target to form the volume data, detect the position and the orientation of the ultrasound probe and target position/orientation information during transmission and reception indicating a position and an orientation of the treatment target during the ultrasound transmission and reception, the treatment tool position/orientation detection unit may be configured to, based on a camera image during treatment obtained by imaging a treatment tool detection mark attached to the treatment tool and the target detection mark with the camera during the treatment on the treatment target, detect the position and the orientation of the treatment tool and target position/orientation information during treatment indicating the position and the orientation of the treatment target during the treatment on the treatment target, and the reconstruction processing unit may be configured to form the reconstructed ultrasound image with reference to a position and an orientation of the treatment tool corrected based on a difference between the target position/orientation information during transmission and reception and the target position/orientation information during treatment.

The display controller may be configured to superimpose and display the reconstructed ultrasound image on a camera image during treatment obtained by imaging the treatment target with a camera during the treatment on the treatment target.

The display controller may be configured to display the reconstructed ultrasound image at a distal end position of the treatment tool in the camera image during treatment.

The reconstruction processing unit may be configured to form a two-dimensional ultrasound image as the reconstructed ultrasound image showing a cross section specified based on the position and the orientation of the treatment tool in the data space of the volume data, from the volume data.

The treatment support apparatus may further comprise: a two-dimensional ultrasound image correction unit configured to correct the two-dimensional ultrasound image so that distortion of the two-dimensional ultrasound image is reduced in a case in which the two-dimensional ultrasound image is viewed from a camera that images the treatment target during the treatment on the treatment target, in which the display controller is configured to superimpose and display the corrected two-dimensional ultrasound image on a camera image during treatment obtained by imaging the treatment target with the camera during the treatment on the treatment target.

The reconstruction processing unit may be configured to form a three-dimensional ultrasound image as the reconstructed ultrasound image from the volume data.

The reconstruction processing unit may be configured to form the three-dimensional ultrasound image by performing a rendering process on the volume data along a ray parallel to an optical axis direction of a camera that images the treatment target during the treatment on the treatment target.

The display controller may be configured to further superimpose and display scale gradations indicating dimensions at positions near the treatment tool on the camera image during treatment.

A treatment support program disclosed in the present specification causes a computer to function as: a probe position/orientation detection unit configured to detect a position and an orientation of an ultrasound probe; a volume data acquisition unit configured to acquire volume data that is formed based on a reception signal obtained by transmitting and receiving ultrasound with the ultrasound probe to and from a treatment target prior to treatment on the treatment target, the volume data having coordinate information indicating each position of the volume data in a data space of the volume data based on the position and the orientation of the ultrasound probe; a treatment tool position/orientation detection unit configured to specify a position and an orientation of a treatment tool for performing the treatment on the treatment target in the data space of the volume data in a case in which the treatment on the treatment target is performed; a reconstruction processing unit configured to form a reconstructed ultrasound image from the volume data with reference to the position and the orientation of the treatment tool in the data space of the volume data; and a display controller configured to display the reconstructed ultrasound image on a display unit.

With the treatment support apparatus disclosed in the present specification, the operator can check the internal structure of the treatment target near the location in which the operator intends to perform the treatment with the treatment tool.

### BRIEF DESCRIPTION OF THE DRAWINGS

FIG. 1 is a schematic configuration diagram of a treatment support system according to the present embodiment.

FIG. 2 is a conceptual diagram showing an endoscopic camera, an ultrasound probe, and a treatment target.

FIG. 3 is a conceptual diagram showing the endoscopic camera, a treatment tool, and the treatment target.

FIG. 4 is a schematic configuration diagram of an ultrasound diagnostic apparatus according to the present embodiment.

FIG. 5 is a diagram showing an example of a camera image during transmission and reception.

FIG. 6 is a conceptual diagram showing a concept of a volume data formation process.

FIG. 7 is a diagram showing an example of a camera image during treatment.

FIG. 8 is a conceptual diagram showing a concept of a first reconstruction process.

FIG. 9 is a conceptual diagram showing a concept of a second reconstruction process.

FIG. 10 is a diagram showing a first display example of a reconstructed ultrasound image.

FIG. 11 is a diagram showing a second display example of the reconstructed ultrasound image.

FIG. 12 is a diagram showing a third display example of the reconstructed ultrasound image.

FIG. 13 is a diagram showing a display example of the reconstructed ultrasound image and grids.

FIG. 14 is a flowchart showing a flow of a process before the treatment of the ultrasound diagnostic apparatus according to the present embodiment.

FIG. 15 is a flowchart showing a flow of a process during the treatment of the ultrasound diagnostic apparatus according to the present embodiment.

### DESCRIPTION OF THE PREFERRED EMBODIMENTS

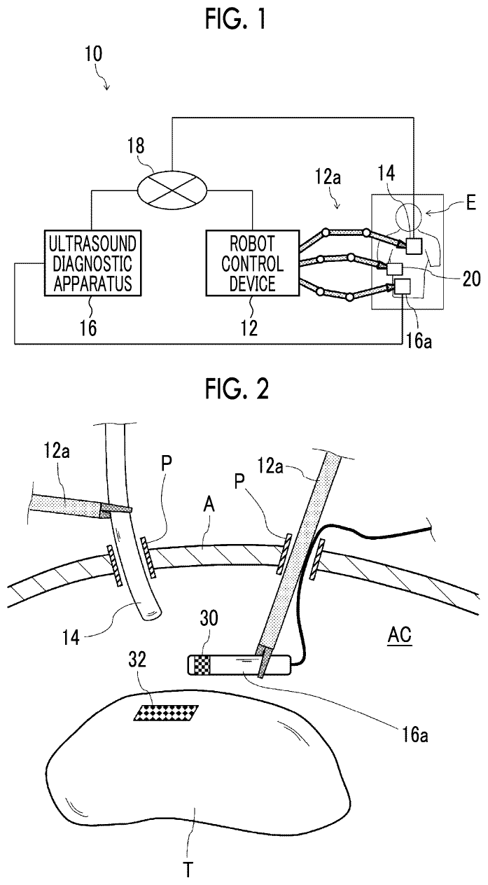

FIG. 1 is a schematic configuration diagram of a treatment support system 10 according to the present embodiment. The treatment support system 10 includes a robot control device 12 including a plurality of robot arms 12a, an endoscope 14, and an ultrasound diagnostic apparatus 16 as a treatment support apparatus including an ultrasound probe 16a. The robot control device 12, the endoscope 14, and the ultrasound diagnostic apparatus 16 are connected to each other via a communication line 18 such as a wide area network (WAN) or a local area network (LAN) so that communication can be performed.

The robot control device 12 includes, in addition to the plurality of robot arms 12a, a processor configured by a central processing unit (CPU) and the like, a communication interface configured by a network adapter and the like, and an input interface configured by a surgeon console and the like. The processor of the robot control device 12 controls the robot arm 12a in accordance with an instruction from an operator such as a doctor. The operator may directly input the instruction to the robot control device 12 through the input interface of the robot control device 12, or may remotely input the instruction through the communication line 18.

The robot arm 12a holds the endoscope 14 and the ultrasound probe 16a. Further, the robot arm 12a holds a treatment tool 20 (for example, scissors or forceps) for performing the treatment. That is, the robot control device 12 controls a position and an orientation of the endoscope 14, a position and an orientation of the ultrasound probe 16a, and a position and an orientation of the treatment tool 20. The robot control device 12 performs the treatment (for example, surgery) on a subject E using the treatment tool 20 while using the endoscope 14 or the ultrasound probe 16a.

The endoscope 14 includes, in addition to a lens and an image sensor, a processor configured by a CPU and the like, a communication interface configured by a network adapter and the like, and the like. The endoscope 14 is a camera that images the inside of the body cavity of the subject E. An endoscopic image is formed by the image sensor of the endoscope 14, and the endoscopic image is transmitted to the ultrasound diagnostic apparatus 16 through the communication interface of the endoscope 14. In addition, the endoscope 14 may be directly connected to the ultrasound diagnostic apparatus 16 through a cable or the like without going through the communication line 18, and the endoscopic image may be directly transmitted to the ultrasound diagnostic apparatus 16.

FIG. 2 is a conceptual diagram showing the endoscope 14, the ultrasound probe 16a, and a treatment target T (organ in the example of FIG. 2). In the present embodiment, it is assumed that laparoscopic surgery is performed on the subject E. FIG. 2 shows a state in which the ultrasound probe 16a is inserted into an abdominal cavity AC prior to the laparoscopic surgery. A small hole is made in an abdomen A of the subject E, and a sleeve (port) P is attached to the hole. The abdominal cavity AC is inflated with gas, and the endoscope 14 and the ultrasound probe 16a are inserted into the abdominal cavity AC from the sleeve P. In the present embodiment, the ultrasound probe 16a is a drop-in type probe that is inserted into the body cavity (in the example of FIG. 2, the abdominal cavity). As described above, the endoscope 14 and the ultrasound probe 16a are held by the robot arm 12a, and the robot control device 12 controls the positions and the orientations of the endoscope 14 and the ultrasound probe 16a.

A probe detection mark 30 is attached to the ultrasound probe 16a. The probe detection mark 30 is a mark for detecting the position and the orientation of the ultrasound probe 16a. The position and the orientation of the ultrasound probe 16a can be detected by imaging the probe detection mark 30 with the endoscope 14 to acquire an endoscopic image, and analyzing an image of the probe detection mark 30 shown in the endoscopic image. Examples of the probe detection mark 30 include an augmented reality (AR) marker.

In addition, a target detection mark 32 is attached to the treatment target T. The target detection mark 32 is a mark for detecting the position and the orientation of the treatment target T. The position and the orientation of the target detection mark 32 can be detected by imaging the target detection mark 32 with the endoscope 14 to acquire the endoscopic image, and analyzing an image of the target detection mark 32 shown in the endoscopic image. Examples of the target detection mark 32 include an AR marker. The target detection mark 32 has a different pattern from the probe detection mark 30.

Although details will be described later, in the present embodiment, before the treatment on the treatment target T, the ultrasound is transmitted and received to and from the treatment target T, and the volume data representing the treatment target T is formed based on a reception signal obtained by the transmission and reception.

In the present specification, a case in which the ultrasound is transmitted and received to and from the treatment target T for forming the volume data in this way is expressed as “ultrasound transmission and reception”.

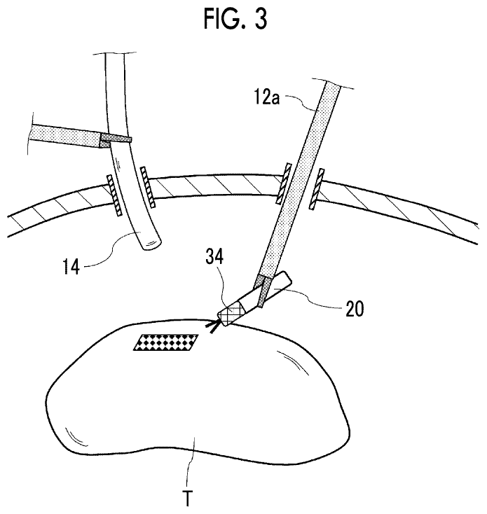

FIG. 3 is a conceptual diagram showing the endoscope 14, the treatment tool 20, and the treatment target T. As shown in FIG. 3, in a case in which the treatment on the treatment target T is performed, the treatment tool 20 is held by the robot arm 12a instead of the ultrasound probe 16a. Then, the treatment tool 20 is inserted into the abdominal cavity AC from the sleeve P, and the operator controls the robot arm 12a to perform the treatment on the treatment target T using the treatment tool 20.

A treatment tool detection mark 34 is attached to the treatment tool 20. The treatment tool detection mark 34 is a mark for detecting the position and the orientation of the treatment tool 20. The position and the orientation of the treatment tool 20 can be detected by imaging the treatment tool detection mark 34 with the endoscope 14 to acquire the endoscopic image, and analyzing an image of the treatment tool detection mark 34 shown in the endoscopic image. Examples of the treatment tool detection mark 34 include an AR marker. The treatment tool detection mark 34 has a different pattern from the probe detection mark 30 and the target detection mark 32.

In the present specification, a period before the treatment on the treatment target T is performed by the treatment tool 20, but in a case in which the treatment on the treatment target T is about to be performed (in other words, after the volume data is formed and before the treatment on the treatment target T is performed), and a period from the start of the treatment on the treatment target T to the completion of the treatment are expressed as “during the treatment on the treatment target T”.

A procedure of the treatment on the treatment target T will be described here. First, during the ultrasound transmission and reception, the ultrasound is transmitted to and received from the treatment target T by the ultrasound probe 16a inserted into the abdominal cavity AC. In this manner, an ultrasound tomographic image is formed in the ultrasound diagnostic apparatus 16. In the present embodiment, a plurality of ultrasound tomographic images are formed by transmitting and receiving the ultrasound while moving the ultrasound probe 16a in a direction substantially perpendicular to an ultrasound transmission/reception surface. The ultrasound diagnostic apparatus 16 forms the volume data, which is three-dimensional ultrasound data, from the plurality of ultrasound tomographic images. In addition, during the ultrasound transmission and reception, the endoscope 14 images the probe detection mark 30 and the target detection mark 32 to form the endoscopic image. In the present specification, the endoscopic image is referred to as a camera image during transmission and reception. The camera image during transmission and reception includes the image of the probe detection mark 30 and the image of the target detection mark 32. In particular, the image of the probe detection mark 30 is information indicating the position and the orientation of the ultrasound probe 16a during the ultrasound transmission and reception, and the image of the target detection mark 32 is information indicating the position and the orientation of the treatment target T (specifically, a surface thereof) during the ultrasound transmission and reception. The camera image during transmission and reception is transmitted from the endoscope 14 to the ultrasound diagnostic apparatus 16.

Then, the treatment on the treatment target T is started by the operator. In order to perform the treatment on the treatment target T, the treatment tool 20 is held by the robot arm 12a instead of the ultrasound probe 16a, and the treatment tool 20 is inserted into the abdominal cavity AC. The endoscope 14 images the treatment tool detection mark 34 and the target detection mark 32 to form the endoscopic image. In the present specification, the endoscopic image formed by the endoscope 14 during the treatment on the treatment target T is referred to as a camera image during treatment. The camera image during treatment includes the image of the treatment tool detection mark 34 and the image of the target detection mark 32. In particular, the image of the treatment tool detection mark 34 is information indicating the position and the orientation of the treatment tool 20 during the treatment on the treatment target T (in other words, at the current point in time), and the image of the target detection mark 32 is information indicating the position and the orientation of the treatment target T during the treatment on the treatment target T. The camera image during treatment is transmitted from the endoscope 14 to the ultrasound diagnostic apparatus 16.

Here, it is desirable that the operator can understand an internal structure of the treatment target T, particularly, an internal structure of the treatment target T near the treatment tool, during the treatment on the treatment target T. The treatment support system 10 supports the operator in this regard.

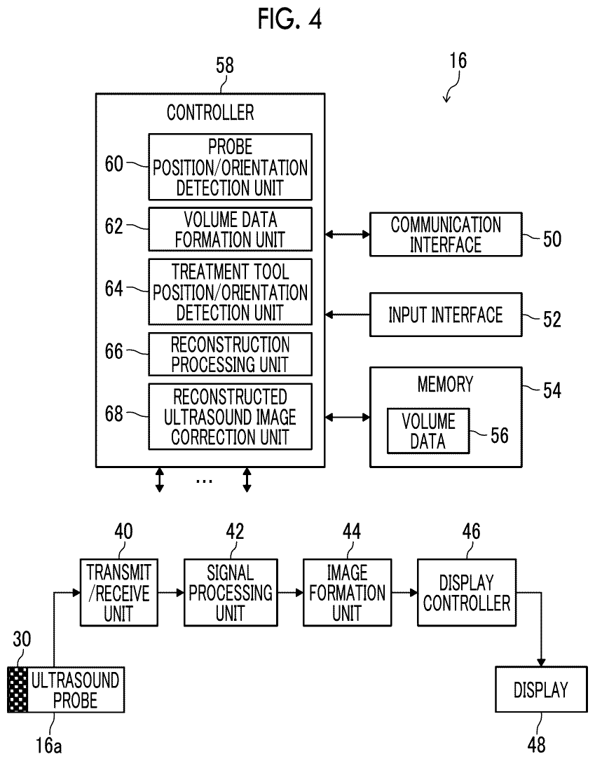

FIG. 4 is a schematic configuration diagram of the ultrasound diagnostic apparatus 16. The ultrasound diagnostic apparatus 16 is a medical apparatus installed in medical institutions such as a hospital.

The ultrasound probe 16a is a device that transmits and receives the ultrasound to and from the treatment target T of the subject E. The ultrasound probe 16a includes a transducer array consisting of a plurality of transducer elements that transmit and receive the ultrasound to and from the treatment target T. In the ultrasound probe 16a, the transducer array is formed of the plurality of transducer elements arranged in one row. In a case in which a transmission signal is supplied to each transducer element from a transmit/receive unit 40, which will be described later, each transducer element generates the ultrasound.

In this way, the probe detection mark 30 is attached to the ultrasound probe 16a.

The transmit/receive unit 40 transmits the transmission signal to the ultrasound probe 16a (specifically, each transducer element of the transducer array) under control of a controller 58 described later. In addition, the transmit/receive unit 40 receives the reception signal from each transducer element that has received the reflected wave reflected by the treatment target T. The transmit/receive unit 40 includes an adder and a plurality of delay elements corresponding to the respective transducer elements, and performs, using the adder and the plurality of delay elements, phase-aligned summation (delay-and-sum) processing in which the reception signals from the respective transducer elements are phase-aligned and added. As a result, a reception beam signal is formed in which pieces of information indicating a signal intensity of the reflected wave reflected by the treatment target T are arranged in a depth direction of the treatment target T.

The signal processing unit 42 executes various kinds of signal processing including filter processing of applying a bandpass filter, detection processing, and the like, on the reception beam signal from the transmit/receive unit 40.

The image formation unit 44 forms the ultrasound tomographic image (B-mode image) representing the cross section (particularly, the ultrasound transmission/reception surface) of the treatment target T based on the reception beam signal on which the signal processing has been performed by the signal processing unit 42.

The display controller 46 performs control to display various images including the ultrasound tomographic image formed by the image formation unit 44 on a display 48.

The display 48 as a display unit is, for example, a display device configured by a liquid crystal display, an organic electroluminescence (EL), or the like.

The transmit/receive unit 40, the signal processing unit 42, the image formation unit 44, and the display controller 46 provided in the ultrasound diagnostic apparatus 16 are configured by a processor. The processor includes at least one of a general-purpose processing device (for example, a CPU) or a dedicated processing device (for example, a graphics processing unit (GPU), an application-specific integrated circuit (ASIC), a field-programmable gate array (FPGA), or a programmable logic device). The processor need not be configured by a single processing device but rather may be configured by cooperation of a plurality of processing devices present at physically separated positions. Further, each of the above-described units may be implemented by cooperation of hardware such as a processor, and software.

A communication interface 50 is configured by, for example, a network adapter. The communication interface 50 exhibits a function of communicating with other devices (particularly, the robot control device 12 and the endoscope 14) via the communication line 18. In particular, in the present embodiment, the communication interface 50 receives the endoscopic image from the endoscope 14 and receives position/orientation information indicating the position and the orientation of each robot arm 12a from the robot control device 12.

An input interface 52 is configured by, for example, a button, a trackball, or a touch panel. The input interface 52 is used to input an instruction of the operator, who uses the ultrasound diagnostic apparatus 16, to the ultrasound diagnostic apparatus 16.

A memory 54 includes a hard disk drive (HDD), a solid-state drive (SSD), an embedded Multi Media Card (eMMC), a read-only memory (ROM), a random-access memory (RAM), or the like. The memory 54 stores a treatment support program for operating each unit of the ultrasound diagnostic apparatus 16. The treatment support program can also be stored in a non-transitory computer-readable storage medium such as a Universal Serial Bus (USB) memory or a CD-ROM. The ultrasound diagnostic apparatus 16 can read the treatment support program from such a storage medium to execute the treatment support program. Since the ultrasound diagnostic apparatus 16 reads the treatment support program to exhibit functions described later, the ultrasound diagnostic apparatus 16 can be said to be a computer program product.

In addition, as shown in FIG. 4, volume data 56 is stored in the memory 54. As described above, the volume data 56 is formed by transmitting and receiving the ultrasound to and from the treatment target T prior to the treatment on the treatment target T. The volume data 56 will be described in detail later.

The controller 58 includes at least one of a general-purpose processor (such as a CPU) or a dedicated processor (such as a GPU, an ASIC, an FPGA, or a programmable logic device). The controller 58 need not be configured by a single processing device but may be configured by cooperation of a plurality of processing devices present at physically separated positions. The controller 58 controls the units of the ultrasound diagnostic apparatus 16. In addition, as shown in FIG. 4, the controller 58 exhibits functions as a probe position/orientation detection unit 60, a volume data formation unit 62, a treatment tool position/orientation detection unit 64, a reconstruction processing unit 66, and a reconstructed ultrasound image correction unit 68 in accordance with the treatment support program stored in the memory 54.

The probe position/orientation detection unit 60 detects the position and the orientation of the ultrasound probe 16a during the ultrasound transmission and reception based on the camera image during transmission and reception that is formed by the endoscope 14. In the present embodiment, the volume data 56 is composed of the plurality of ultrasound tomographic images, and the probe position/orientation detection unit 60 detects the position and the orientation of the ultrasound probe 16a at a timing at which each ultrasound tomographic image forming the volume data 56 is formed (in other words, during the ultrasound transmission and reception for the ultrasound tomographic image).

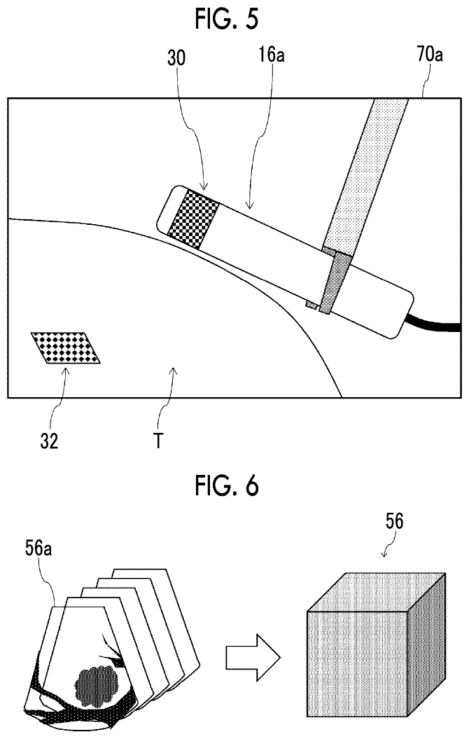

FIG. 5 is a diagram showing an example of a camera image during transmission and reception 70a. As described above, the camera image during transmission and reception 70a includes the image of the probe detection mark 30 indicating the position and the orientation of the ultrasound probe 16a during the ultrasound transmission and reception. The probe position/orientation detection unit 60 detects the position and the orientation of the ultrasound probe 16a during the ultrasound transmission and reception in a camera coordinate system of the endoscope 14 by analyzing the image of the probe detection mark 30 in the camera image during transmission and reception 70a. Since a known method can be used as a method of detecting the position and the orientation of the ultrasound probe 16a in the camera coordinate system from the image of the probe detection mark 30 included in the camera image during transmission and reception 70a, a detailed description thereof will be omitted here.

Next, the probe position/orientation detection unit 60 converts position/orientation information (hereinafter, simply referred to as probe position/orientation information) indicating the position and the orientation of the ultrasound probe 16a in the camera coordinate system into position/orientation information in a robot coordinate system. The robot coordinate system is a coordinate system that is recognized by the robot control device 12 and that is used to represent the position and the orientation of the robot arm 12a.

Since the ultrasound probe 16a is held by the robot arm 12a, a position/orientation relationship between the ultrasound probe 16a and the robot arm 12a is fixed (not changed). In the present specification, a fixed position/orientation relationship between the ultrasound probe 16a and the robot arm 12a is referred to as an arm-probe position/orientation relationship. The arm-probe position/orientation relationship can be, for example, represented by a vector in the robot coordinate system. The probe position/orientation detection unit 60 converts the probe position/orientation information in the camera coordinate system into the position/orientation information in the robot coordinate system in accordance with an arm-probe position/orientation relationship.

Specifically, the probe position/orientation detection unit 60 acquires the position/orientation information of the robot arm 12a that holds the ultrasound probe 16a in the robot coordinate system and the information indicating the arm-probe position/orientation relationship from the robot control device 12. The information indicating the arm-probe position/orientation relationship may be input to the robot control device 12 by, for example, the operator who causes the robot arm 12a to hold the ultrasound probe 16a. The probe position/orientation detection unit 60 can obtain the probe position/orientation information in the robot coordinate system by using the position/orientation information of the robot arm 12a in the robot coordinate system and the arm-probe position/orientation relationship. Here, by aligning the respective axial directions of the camera coordinate system and the robot coordinate system, a transformation vector from the camera coordinate system to the robot coordinate system can be obtained based on the probe position/orientation information in the camera coordinate system and the robot coordinate system. The probe position/orientation detection unit 60 converts the probe position/orientation information in the camera coordinate system into the position/orientation information in the robot coordinate system by using the transformation vector.

The probe position/orientation detection unit 60 can also obtain the probe position/orientation information in the robot coordinate system by simply using the position/orientation information of the robot arm 12a in the robot coordinate system and the arm-probe position/orientation relationship without analyzing the image of the probe detection mark 30 in the camera image during transmission and reception 70a. However, the position and the orientation of the ultrasound probe 16a can be detected with higher accuracy in a case in which the position and the orientation are detected by using the probe detection mark 30.

The probe position/orientation detection unit 60 stores the probe position/orientation information in the memory 54 in association with each ultrasound tomographic image in a case in which the ultrasound tomographic image is formed.

The probe position/orientation detection unit 60 may further detect the position and the orientation of the treatment target T during the ultrasound transmission and reception.

As described above, the camera image during transmission and reception 70a also includes the image of the target detection mark 32 indicating the position and the orientation of the treatment target T during the ultrasound transmission and reception. The probe position/orientation detection unit 60 detects the position and the orientation of the treatment target T during the ultrasound transmission and reception in the camera coordinate system of the endoscope 14 by analyzing the image of the target detection mark 32 in the camera image during transmission and reception 70a. Since a known method can be used as a method of detecting the position and the orientation of the treatment target T in the camera coordinate system from the image of the target detection mark 32 included in the camera image during transmission and reception 70a, a detailed description thereof will be omitted here.

Next, similarly to the ultrasound probe 16a, the probe position/orientation detection unit 60 converts the position/orientation information indicating the position and the orientation of the treatment target T in the camera coordinate system into the position/orientation information in the robot coordinate system in accordance with the arm-probe position/orientation relationship. In the present specification, information indicating the position and the orientation of the treatment target T detected based on the camera image during transmission and reception 70a is referred to as target position/orientation information during transmission and reception. The probe position/orientation detection unit 60 stores the target position/orientation information during transmission and reception in the memory 54 in association with each ultrasound tomographic image.

FIG. 6 is a conceptual diagram showing a concept of a volume data formation process performed by the volume data formation unit 62. The volume data formation unit 62 as a volume data acquisition unit forms the volume data 56 based on the reception signal obtained by transmitting and receiving the ultrasound with the ultrasound probe 16a to and from the treatment target T, prior to the treatment on the treatment target T. In the present embodiment, the volume data formation unit 62 forms the volume data 56 based on a plurality of ultrasound tomographic images 56a. Since a known method can be used as a method of forming the volume data 56 based on the plurality of ultrasound tomographic images 56a, a detailed description thereof will be omitted here.

As described above, the probe position/orientation information (in the present embodiment, in the robot coordinate system) at the time of forming the ultrasound tomographic image 56a is associated with each ultrasound tomographic image 56a. Each position (coordinate) in the ultrasound tomographic image 56a can be specified based on the position and the orientation of the ultrasound probe 16a in a case in which the ultrasound tomographic image 56a is formed. That is, it can be said that coordinate information indicating the position of each ultrasound tomographic image 56a is attached to each ultrasound tomographic image 56a. Therefore, the volume data 56 composed of the plurality of ultrasound tomographic images 56a also has coordinate information indicating each position of the volume data 56. In the present embodiment, the coordinate information of the volume data 56 is coordinate information in the robot coordinate system.

In the present embodiment, the volume data 56 is composed of the plurality of ultrasound tomographic images 56a, but, in a case in which a 2D array probe in which the transducer elements are arranged in a two-dimensional manner is used as the ultrasound probe 16a, the volume data formation unit 62 may directly form the volume data 56 based on three-dimensional reception signals from the ultrasound probe 16a. Even in this case, each position (coordinate) in the volume data 56 can be specified based on the position and the orientation of the ultrasound probe 16a in a case in which the volume data 56 is formed. That is, in this case as well, the coordinate information indicating each position of the volume data 56 is added to the volume data 56.

The treatment tool position/orientation detection unit 64 specifies the position and the orientation of the treatment tool 20 in the data space (robot coordinate system in the present embodiment) of the volume data 56 based on the camera image during treatment, which is formed by the endoscope 14, in a case in which the treatment is performed on the treatment target T.

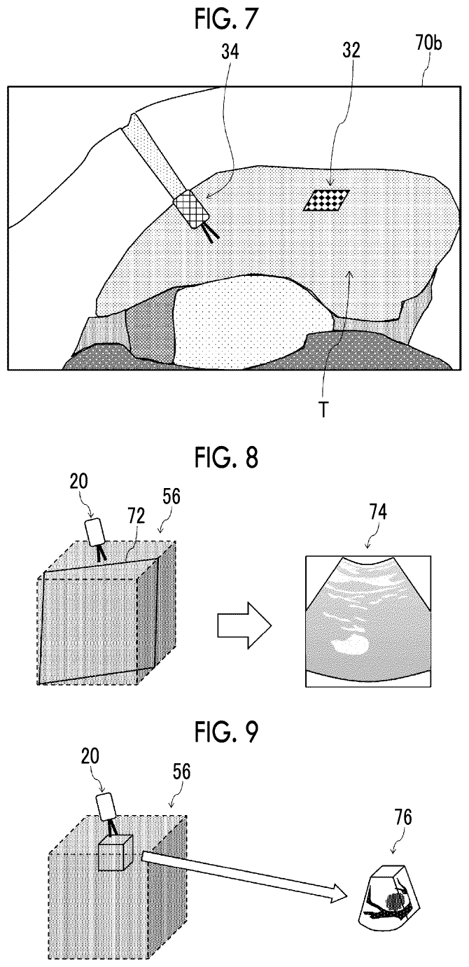

FIG. 7 is a diagram showing an example of a camera image during treatment 70b. As described above, the camera image during treatment 70b includes the image of the treatment tool detection mark 34 indicating the position and the orientation of the treatment tool 20 during the treatment on the treatment target T. The treatment tool position/orientation detection unit 64 analyzes the image of the treatment tool detection mark 34 in the camera image during treatment 70b to detect the position and the orientation of the treatment tool 20 during the treatment on the treatment target T in the camera coordinate system of the endoscope 14. Since a known method can be used as a method of detecting the position and the orientation of the treatment tool 20 in the camera coordinate system from the image of the treatment tool detection mark 34 included in the camera image during treatment 70b, a detailed description thereof will be omitted here.

Next, the treatment tool position/orientation detection unit 64 converts position/orientation information (hereinafter, simply referred to as treatment tool position/orientation information) indicating the position and the orientation of the treatment tool 20 in the camera coordinate system into position/orientation information in the robot coordinate system.

Since the treatment tool 20 is held by the robot arm 12a, a position/orientation relationship between the treatment tool 20 and the robot arm 12a is fixed (not changed). In the present specification, a fixed position/orientation relationship between the treatment tool 20 and the robot arm 12a is referred to as an arm-treatment tool position/orientation relationship. The arm-treatment tool position/orientation relationship can be represented by, for example, a vector in the robot coordinate system. The treatment tool position/orientation detection unit 64 converts the treatment tool position/orientation information in the camera coordinate system into the position/orientation information in the robot coordinate system in accordance with the arm-treatment tool position/orientation relationship.

Specifically, the treatment tool position/orientation detection unit 64 acquires the position/orientation information of the robot arm 12a that holds the treatment tool 20 in the robot coordinate system and the information indicating the arm-treatment tool position/orientation relationship from the robot control device 12. The information indicating the arm-treatment tool position/orientation relationship may be input to the robot control device 12 by, for example, the operator who causes the robot arm 12a to hold the treatment tool 20. The treatment tool position/orientation detection unit 64 can obtain the treatment tool position/orientation information in the robot coordinate system by the position/orientation information of the robot arm 12a in the robot coordinate system and the arm-treatment tool position/orientation relationship. Here, by aligning the respective axial directions of the camera coordinate system and the robot coordinate system, a transformation vector from the camera coordinate system to the robot coordinate system can be obtained based on the position/orientation information of the treatment tool 20 in the camera coordinate system and the robot coordinate system. The treatment tool position/orientation detection unit 64 converts the treatment tool position/orientation information in the camera coordinate system into the position/orientation information in the robot coordinate system by using the transformation vector.

The treatment tool position/orientation detection unit 64 can also obtain the treatment tool position/orientation information in the robot coordinate system by simply using the position/orientation information of the robot arm 12a in the robot coordinate system and the arm-treatment tool position/orientation relationship without analyzing the image of the treatment tool detection mark 34 in the camera image during treatment 70b. However, the position and the orientation of the treatment tool 20 can be detected with higher accuracy in a case in which the position and the orientation are detected by using the treatment tool detection mark 34.

The treatment tool position/orientation detection unit 64 may further detect the position and the orientation of the treatment target T during the treatment on the treatment target T.

As described above, the camera image during treatment 70b also includes the image of the target detection mark 32 indicating the position and the orientation of the treatment target T during the treatment on the treatment target T. The treatment tool position/orientation detection unit 64 analyzes the image of the target detection mark 32 in the camera image during treatment 70b to detect the position and the orientation of the treatment target T during the treatment on the treatment target T in the camera coordinate system of the endoscope 14.

Next, similarly to the treatment tool 20, the treatment tool position/orientation detection unit 64 converts the position/orientation information indicating the position and the orientation of the treatment target T in the camera coordinate system into the position/orientation information in the robot coordinate system in accordance with the arm-treatment tool position/orientation relationship. In the present specification, information indicating the position and the orientation of the treatment target T detected based on the camera image during treatment 70b is referred to as target position/orientation information during treatment.

The reconstruction processing unit 66 forms the reconstructed ultrasound image from the volume data 56 with reference to the position and the orientation of the treatment tool 20 in the data space of the volume data 56 detected by the treatment tool position/orientation detection unit 64. As described above, the volume data 56 has the coordinate information in the robot coordinate system indicating each position of the volume data 56, and the position and the orientation of the treatment tool 20 in the robot coordinate system are detected by the treatment tool position/orientation detection unit 64. This means that the reconstruction processing unit 66 detects the position and the orientation of the treatment tool 20 in the data space (actually, in the robot coordinate system) of the volume data 56.

As described above, in a case in which the probe position/orientation detection unit 60 detects the target position/orientation information during transmission and reception, and the treatment tool position/orientation detection unit 64 detects the target position/orientation information during treatment, the reconstruction processing unit 66 may correct the position and the orientation of the treatment tool 20 based on a difference between the target position/orientation information during transmission and reception and the target position/orientation information during treatment, and may form the reconstructed ultrasound image with reference to the corrected position and orientation of the treatment tool 20. Specifically, the reconstruction processing unit 66 moves the position and the orientation of the treatment tool 20 and the position and the orientation of the treatment target T indicated by the target position/orientation information during treatment while maintaining a relative relationship therebetween such that the position and the orientation of the treatment target T indicated by the target position/orientation information during transmission and reception and the position and the orientation of the treatment target T indicated by the target position/orientation information during treatment match each other. The position and the orientation of the treatment tool 20 after the movement are the position and the orientation of the treatment tool 20 after the correction.

FIG. 8 is a conceptual diagram showing a concept of a first reconstruction process performed by the reconstruction processing unit 66. The reconstruction processing unit 66 can form a two-dimensional ultrasound image 74 as the reconstructed ultrasound image from the volume data 56. In this case, the reconstruction processing unit 66 specifies a reconstructed cross section 72 in the volume data 56 based on the position and the orientation of the treatment tool 20 in the data space of the volume data 56. For example, a plane at the distal end position (cutting edge position) of the treatment tool 20 and parallel to a predetermined direction (for example, an opening direction of scissors) of the treatment tool 20 is specified as the reconstructed cross section 72. Since a position/orientation relationship between the distal end position of the treatment tool 20 and the treatment tool detection mark 34 is fixed, the distal end position of the treatment tool 20 and the direction of the scissors can be specified based on the position and the orientation of the treatment tool detection mark 34. Then, the reconstruction processing unit 66 forms the two-dimensional ultrasound image 74 showing the specified reconstructed cross section 72 by reconstructing the volume data 56 as the reconstructed ultrasound image. Since a known method can be used as a method of forming the two-dimensional ultrasound image 74 by the reconstruction process, a detailed description thereof will be omitted here.

FIG. 9 is a conceptual diagram showing a concept of a second reconstruction process performed by the reconstruction processing unit 66. The reconstruction processing unit 66 can form a three-dimensional ultrasound image 76 as the reconstructed ultrasound image from the volume data 56. In this case, the reconstruction processing unit 66 cuts out a part of the volume data 56 based on the position and the orientation of the treatment tool 20 (for example, a cutting edge of the treatment tool 20) in the data space of the volume data 56, and performs a rendering process on the part of the volume data 56 to form the three-dimensional ultrasound image 76.

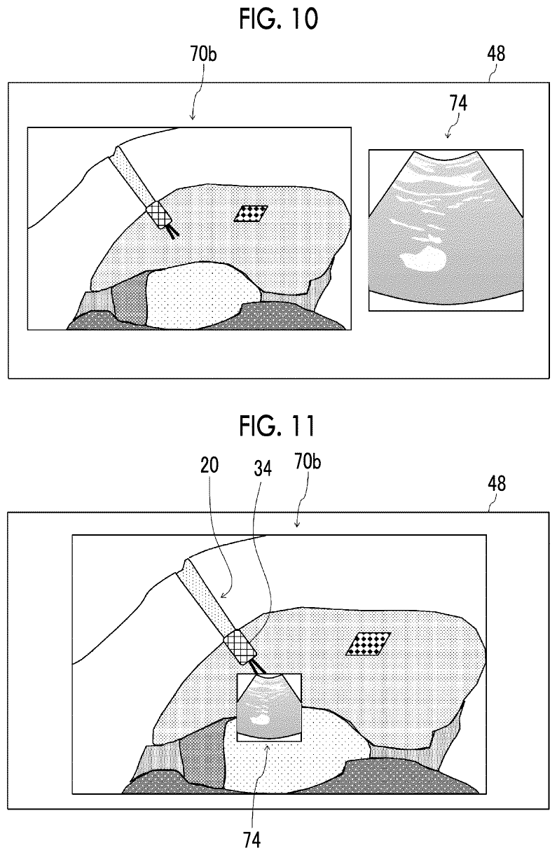

The display controller 46 displays the reconstructed ultrasound image formed by the reconstruction processing unit 66 on the display 48. FIG. 10 is a diagram showing a first display example of the reconstructed ultrasound image. As shown in FIG. 10, the display controller 46 may display the camera image during treatment 70b and the reconstructed ultrasound image side by side. The camera image during treatment 70b need not be displayed together with the reconstructed ultrasound image. In the example of FIG. 10, although the two-dimensional ultrasound image 74 is displayed as the reconstructed ultrasound image, the three-dimensional ultrasound image 76 may be displayed instead of or in addition to the two-dimensional ultrasound image 74.

FIG. 11 is a diagram showing a second display example of the reconstructed ultrasound image. As shown in FIG. 11, the display controller 46 may superimpose and display the two-dimensional ultrasound image 74 on the camera image during treatment 70b, as the reconstructed ultrasound image. In particular, the display controller 46 may display the two-dimensional ultrasound image 74 at the distal end position of the treatment tool 20 in the camera image during treatment 70b. The position of the treatment tool 20 in the camera image during treatment 70b can be specified by detecting the treatment tool detection mark 34.

Since the position and the orientation of the treatment tool 20 change from moment to moment, the reconstruction processing unit 66 may dynamically form the two-dimensional ultrasound image 74 in accordance with the change in the position and the orientation of the treatment tool 20, and the display controller 46 may dynamically change the position at which the two-dimensional ultrasound image 74 is to be displayed, following the change in the distal end position of the treatment tool 20.

As shown in FIG. 11, in a case in which the two-dimensional ultrasound image 74 is superimposed and displayed on the camera image during treatment 70b, the reconstructed ultrasound image correction unit 68 corrects the two-dimensional ultrasound image 74 such that the distortion of the two-dimensional ultrasound image 74 is reduced in a case in which the two-dimensional ultrasound image 74 is viewed from the endoscope 14 that captures the camera image during treatment 70b. Specifically, the reconstructed ultrasound image correction unit 68 performs an affine transformation on the two-dimensional ultrasound image 74 such that the position of the endoscope 14 is located at a viewpoint position in the two-dimensional ultrasound image 74. In addition, the display controller 46 may superimpose and display the corrected two-dimensional ultrasound image 74 on the camera image during treatment 70b.

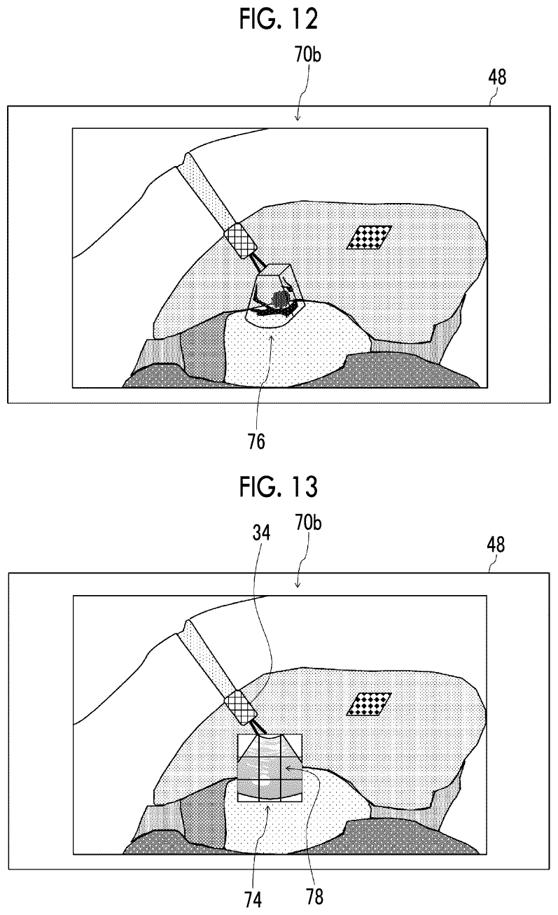

FIG. 12 is a diagram showing a third display example of the reconstructed ultrasound image. As shown in FIG. 12, the display controller 46 may superimpose and display the three-dimensional ultrasound image 76 on the camera image during treatment 70b. In particular, the display controller 46 may display the three-dimensional ultrasound image 76 at the distal end position of the treatment tool 20 in the camera image during treatment 70b.

Here, since the position and the orientation of the treatment tool 20 change from moment to moment, the reconstruction processing unit 66 may dynamically form the three-dimensional ultrasound image 76 in accordance with the change in the position and the orientation of the treatment tool 20, and the display controller 46 may dynamically change the position at which the three-dimensional ultrasound image 76 is to be displayed, following the change in the distal end position of the treatment tool 20.

As shown in FIG. 12, in a case in which the three-dimensional ultrasound image 76 is superimposed and displayed on the camera image during treatment 70b, the reconstruction processing unit 66 may form the three-dimensional ultrasound image 76 by performing a rendering process on the volume data 56 along a ray parallel to an optical axis direction of the endoscope 14 that captures the camera image during treatment 70b.

In addition, as shown in FIG. 13, in a case in which the reconstructed ultrasound image is superimposed and displayed on the camera image during treatment 70b, the display controller 46 may further superimpose and display scale gradations representing the dimensions of the positions near the treatment tool 20 on the camera image during treatment 70b. In the example of FIG. 13, the display controller 46 superimposes and displays grids 78 as the scale gradations on the two-dimensional ultrasound image 74 as the reconstructed ultrasound image. An interval of the grids 78 may represent a predetermined length (for example, 10 mm) in the real space between the positions near the treatment tool 20. A distance on the camera image during treatment 70b corresponding to the predetermined length in the real space between the positions near the treatment tool 20 can be obtained based on a distance in the real space from the endoscope 14 (specifically, the lens) to the treatment tool detection mark 34, which is obtained by detecting the treatment tool detection mark 34, the imaging magnification of the endoscope 14, and the like. In the example of FIG. 13, the grids 78 are superimposed and displayed on the two-dimensional ultrasound image 74 as the reconstructed ultrasound image, but the grids 78 may be superimposed and displayed on the three-dimensional ultrasound image 76.

Here, since the position and the orientation of the treatment tool 20 change from moment to moment, the display controller 46 may dynamically change the position at which the grids 78 are to be displayed, following the change in the position and the orientation of the treatment tool 20. In addition, in a case in which the instruction is given from the operator, the display position of the grids 78 may be fixed regardless of the position and the orientation of the treatment tool 20.

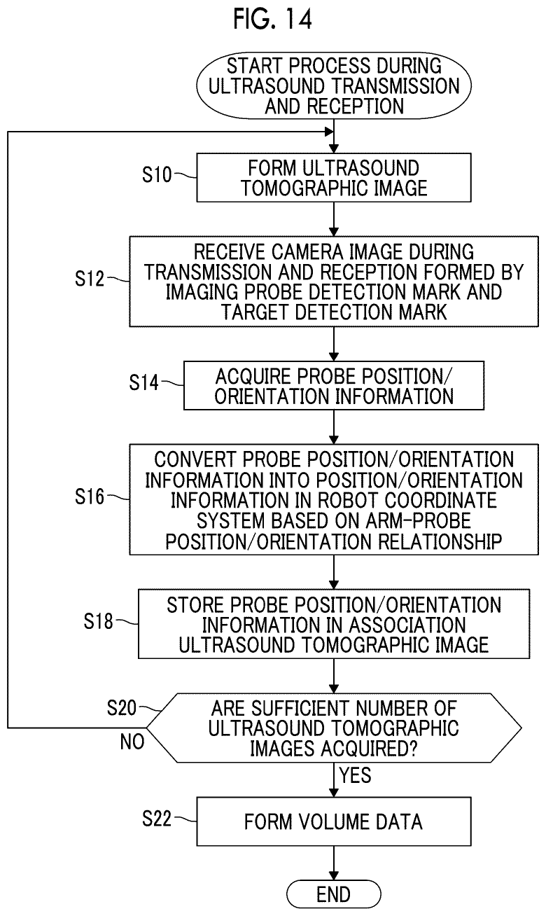

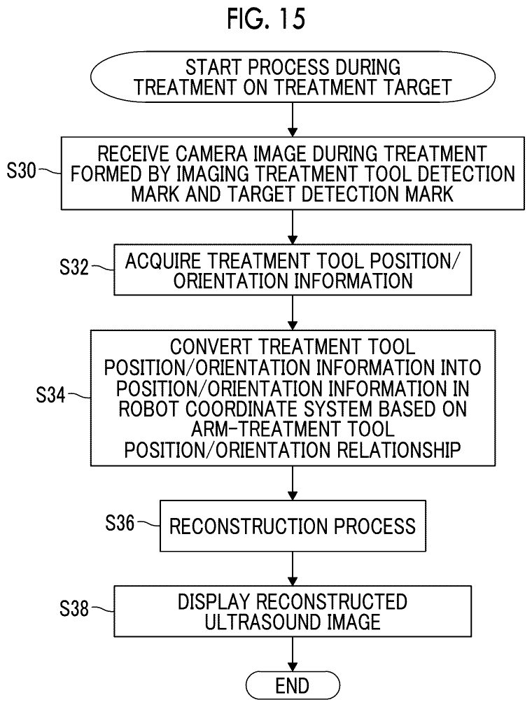

The schematic configuration of the ultrasound diagnostic apparatus 16 as the treatment support apparatus according to the present embodiment is as described above. Hereinafter, a flow of a process of the ultrasound diagnostic apparatus 16 according to the present embodiment will be described with reference to flowcharts shown in FIGS. 14 and 15.

FIG. 14 is a flowchart showing a flow of a processing during the ultrasound transmission and reception in the present embodiment.

In step S10, the ultrasound probe 16a transmits and receives the ultrasound to and from the treatment target T. The image formation unit 44 forms the ultrasound tomographic image 56a for forming the volume data 56 based on the reception signal from the ultrasound probe 16a.

In step S12, the endoscope 14 images the probe detection mark 30 during the ultrasound transmission and reception to form the camera image during transmission and reception 70a. The ultrasound diagnostic apparatus 16 receives the camera image during transmission and reception 70a from the endoscope 14.

In step S14, the probe position/orientation detection unit 60 acquires the probe position/orientation information based on the camera image during transmission and reception 70a received in step S12.

In step S16, the probe position/orientation detection unit 60 converts the probe position/orientation information in the camera coordinate system acquired in step S14 into the probe position/orientation information in the robot coordinate system based on the arm-probe position/orientation relationship.

In step S18, the probe position/orientation detection unit 60 stores the probe position/orientation information of the robot coordinate system acquired in step S16 in the memory 54 in association with the ultrasound tomographic image 56a formed in step S10.

In step S20, the volume data formation unit 62 determines whether or not a sufficient number of ultrasound tomographic images 56a for forming the volume data 56 are acquired. In a case in which a sufficient number of ultrasound tomographic images 56a are not acquired, the process returns to step S10, and the processes of steps S10 to S20 are repeated. In a case in which a sufficient number of ultrasound tomographic images 56a are acquired, the process proceeds to step S22.

In step S22, the volume data formation unit 62 forms the volume data 56 based on the plurality of acquired ultrasound tomographic images 56a, and stores the volume data 56 in the memory 54.

FIG. 15 is a flowchart showing a flow of a process during the treatment on the treatment target T in the present embodiment.

In step S30, the endoscope 14 images the treatment tool detection mark 34 during the treatment on the treatment target T to form the camera image during treatment 70b. The ultrasound diagnostic apparatus 16 receives the camera image during treatment 70b from the endoscope 14.

In step S32, the treatment tool position/orientation detection unit 64 acquires the treatment tool position/orientation information based on the camera image during treatment 70b received in step S30.

In step S34, the treatment tool position/orientation detection unit 64 converts the treatment tool position/orientation information in the camera coordinate system, which is acquired in step S32, into treatment tool position/orientation information in the robot coordinate system based on the arm-treatment tool position/orientation relationship.

In step S36, the reconstruction processing unit 66 forms the reconstructed ultrasound image from the volume data 56 based on the treatment tool position/orientation information in the robot coordinate system (that is, in the data space of the volume data 56) acquired in step S34.

In step S38, the display controller 46 displays the reconstructed ultrasound image formed in step S36 on the display 48.

Although the treatment support apparatus according to the present disclosure has been described above, the treatment support apparatus according to the present disclosure is not limited to the above-described embodiment, and various modifications can be made without departing from the gist of the present disclosure.

For example, in the present embodiment, the ultrasound probe 16a is a drop-in type probe, but the ultrasound probe 16a may be other kinds of probes. The ultrasound probe 16a may be, for example, a probe that is in contact with the body surface of the subject E. In addition, although the camera that images the probe detection mark 30, the target detection mark 32, and the treatment tool detection mark 34 is the endoscope 14, the camera need not be the endoscope 14. For example, the camera may be an extracorporeal camera that is located away from the body surface of the subject E and that images the subject E from the outside.

In addition, in the above-described embodiment, the position/orientation information of each of the ultrasound probe 16a, the treatment target T, and the treatment tool 20 in the camera coordinate system is converted into the position/orientation information in the robot coordinate system, but, in a case in which the position and the orientation of the endoscope 14 do not change between during the ultrasound transmission and during the treatment on the treatment target T, it is not necessary to perform the conversion from the camera coordinate system into the robot coordinate system.

In addition, in the above-described embodiment, the controller 58 of the ultrasound diagnostic apparatus 16 has each of the functions of the probe position/orientation detection unit 60, the volume data formation unit 62, the treatment tool position/orientation detection unit 64, the reconstruction processing unit 66, and the reconstructed ultrasound image correction unit 68, but these functions need not be always exhibited by the ultrasound diagnostic apparatus 16. For example, these functions may be exhibited by a server computer or the like that is connected to the robot control device 12, the endoscope 14, and the ultrasound diagnostic apparatus 16 in a communicable manner. Further, at least one of the above-described functions may be exhibited by the robot control device 12. Furthermore, all of the above-described functions may be exhibited by cooperation of a plurality of devices without being exhibited by one device.

What is claimed is:

1. A treatment support apparatus comprising:
a probe position/orientation detection unit configured to detect a position and an orientation of an ultrasound probe;
a volume data acquisition unit configured to acquire volume data that is formed based on a reception signal obtained by transmitting and receiving ultrasound with the ultrasound probe to and from a treatment target prior to treatment on the treatment target, the volume data having coordinate information indicating each position of the volume data in a data space of the volume data based on the position and the orientation of the ultrasound probe;
a treatment tool position/orientation detection unit configured to specify a position and an orientation of a treatment tool for performing the treatment on the treatment target in the data space of the volume data in a case in which the treatment on the treatment target is performed;
a reconstruction processing unit configured to form a reconstructed ultrasound image from the volume data with reference to the position and the orientation of the treatment tool in the data space of the volume data; and
a display controller configured to display the reconstructed ultrasound image on a display unit.

2. The treatment support apparatus according to claim 1,
wherein the probe position/orientation detection unit is configured to, based on a camera image during transmission and reception obtained by imaging a probe detection mark attached to the ultrasound probe and a target detection mark attached to the treatment target with a camera during ultrasound transmission and reception in which the ultrasound is transmitted to and received from the treatment target to form the volume data, detect the position and the orientation of the ultrasound probe and target position/orientation information during transmission and reception indicating a position and an orientation of the treatment target during the ultrasound transmission and reception,
the treatment tool position/orientation detection unit is configured to, based on a camera image during treatment obtained by imaging a treatment tool detection mark attached to the treatment tool and the target detection mark with the camera during the treatment on the treatment target, detect the position and the orientation of the treatment tool and target position/orientation information during treatment indicating the position and the orientation of the treatment target during the treatment on the treatment target, and
the reconstruction processing unit is configured to form the reconstructed ultrasound image with reference to a position and an orientation of the treatment tool corrected based on a difference between the target position/orientation information during transmission and reception and the target position/orientation information during treatment.

3. The treatment support apparatus according to claim 1,
wherein the display controller is configured to superimpose and display the reconstructed ultrasound image on a camera image during treatment obtained by imaging the treatment target with a camera during the treatment on the treatment target.

4. The treatment support apparatus according to claim 3,
wherein the display controller is configured to display the reconstructed ultrasound image at a distal end position of the treatment tool in the camera image during treatment.

5. The treatment support apparatus according to claim 1,
wherein the reconstruction processing unit is configured to form a two-dimensional ultrasound image as the reconstructed ultrasound image showing a cross section specified based on the position and the orientation of the treatment tool in the data space of the volume data, from the volume data.

6. The treatment support apparatus according to claim 5, further comprising:
a two-dimensional ultrasound image correction unit configured to correct the two-dimensional ultrasound image so that distortion of the two-dimensional ultrasound image is reduced in a case in which the two-dimensional ultrasound image is viewed from a camera that images the treatment target during the treatment on the treatment target,
wherein the display controller is configured to superimpose and display the corrected two-dimensional ultrasound image on a camera image during treatment obtained by imaging the treatment target with the camera during the treatment on the treatment target.

7. The treatment support apparatus according to claim 1,
wherein the reconstruction processing unit is configured to form a three-dimensional ultrasound image as the reconstructed ultrasound image from the volume data.

8. The treatment support apparatus according to claim 7,
wherein the reconstruction processing unit is configured to form the three-dimensional ultrasound image by performing a rendering process on the volume data along a ray parallel to an optical axis direction of a camera that images the treatment target during the treatment on the treatment target.

9. The treatment support apparatus according to claim 3,
wherein the display controller is configured to further superimpose and display scale gradations indicating dimensions at positions near the treatment tool on the camera image during treatment.

10. A non-transitory computer-readable storage medium storing a treatment support program causing a computer to function as:
a probe position/orientation detection unit configured to detect a position and an orientation of an ultrasound probe;
a volume data acquisition unit configured to acquire volume data that is formed based on a reception signal obtained by transmitting and receiving ultrasound with the ultrasound probe to and from a treatment target prior to treatment on the treatment target, the volume data having coordinate information indicating each position of the volume data in a data space of the volume data based on the position and the orientation of the ultrasound probe;
a treatment tool position/orientation detection unit configured to specify a position and an orientation of a treatment tool for performing the treatment on the treatment target in the data space of the volume data in a case in which the treatment on the treatment target is performed;
a reconstruction processing unit configured to form a reconstructed ultrasound image from the volume data with reference to the position and the orientation of the treatment tool in the data space of the volume data; and
a display controller configured to display the reconstructed ultrasound image on a display unit.
